(19) United States
(12) Patent Application Publication
(10) Pub. No.: US 2020/0311989 A1
(43) Pub. Date: Oct. 1, 2020

(54) IMAGE PROCESSING APPARATUS, IMAGE PROCESSING METHOD, AND COMPUTER READABLE MEDIUM

(71) Applicant: CANON KABUSHIKI KAISHA, Tokyo (JP)

(72) Inventors: Osamu Tsujii, Ichikawa-shi (JP); Tetsuo Shimada, Sanjo-shi (JP)

(21) Appl. No.: 16/902,561

(22) Filed: Jun. 16, 2020

Related U.S. Application Data
(63) Continuation of application No. PCT/JP2018/045463, filed on Dec. 11, 2018.

(30) Foreign Application Priority Data
Dec. 18, 2017 (JP) 2017-241732

(51) Int. Cl. G06T 11/00; G06T 15/08; A61B 6/00; A61B 6/03; A61B 6/04

(52) U.S. Cl. CPC G06T 11/008; G06T 11/005; G06T 15/08; G06T 2210/41; A61B 6/032; A61B 6/0435; A61B 6/502

(57) ABSTRACT

Provided is an image processing apparatus including: a first generating unit configured to generate a three-dimensional image with use of an image signal obtained by imaging an imaging target object with a radiation; a second generating unit configured to set a projection reference line in the three-dimensional image, and perform projection processing of projecting pixel values of the three-dimensional image to the projection reference line for each projection angle of a plurality of projection angles about the projection reference line, to thereby generate a plurality of pieces of projection data at the plurality of projection angles; and a third generating unit configured to two-dimensionally arrange the plurality of pieces of projection data based on the plurality of projection angles, to thereby generate a two-dimensional image.

### CROSS-REFERENCE TO RELATED APPLICATIONS

This application is a Continuation of International Patent Application No. PCT/JP2018/045463, filed Dec. 11, 2018, which claims the benefit of Japanese Patent Application No. 2017-241732, filed Dec. 18, 2017, both of which are hereby incorporated by reference herein in their entirety.

### BACKGROUND OF THE INVENTION

### Field of the Invention

The present invention relates to an image processing apparatus configured to process an image obtained by radiation imaging, an image processing method, and a computer readable medium having stored thereon a program for causing a computer to function as the image processing apparatus.

### Description of the Related Art

In the related art, there has been proposed an image processing technology for displaying a three-dimensional image, for example, X-ray CT or MRI. For example, there is known a technology in which, as illustrated in FIG. 9, a human body shown in a display area 920 for items defined in a display area 910 is displayed in a standard manner in an axial, coronal, or sagittal section, and a three-dimensional volume rendering (VR) image is further displayed (for example, Japanese Patent Application Laid-Open No. 2004-181041). Specifically, in Japanese Patent Application Laid-Open No. 2004-181041, a technology for displaying a bull's eye map is proposed. Here, the bull's eye map is a technology for arranging short-axis tomography images perpendicular to a long axis on concentric circles based on three-dimensional image information of a heart to generate a two-dimensional image of the heart, and displaying the two-dimensional image. This bull's eye map is generated for each cardiac function indicating various operations and states of the heart.

The technology described in Japanese Patent Application Laid-Open No. 2004-181041 described above is a technology that is very useful in generating a two-dimensional image of a hollow organ, such as a heart, as an imaging target object. However, the technology described in Japanese Patent Application Laid-Open No. 2004-181041 described above has a problem in that, when a two-dimensional image is to be generated of an organ filled with contents (tissues), such as a breast, as an imaging target object, for example, the two-dimensional image is insufficient as a diagnostic image.

The present invention has been made in view of the above-mentioned problem, and therefore has an object to provide a mechanism with which a two-dimensional image suitable as a diagnostic image can be generated even for an imaging target object filled with contents.

### SUMMARY OF THE INVENTION

According to at least one aspect of the present invention, there is provided an image processing apparatus including: a first generating unit configured to generate a three-dimensional image with use of an image signal obtained by imaging an imaging target object with a radiation; a second generating unit configured to set a projection reference line in the three-dimensional image, and perform projection processing of projecting pixel values of the three-dimensional image to the projection reference line for each projection angle of a plurality of projection angles about the projection reference line, to thereby generate a plurality of pieces of projection data at the plurality of projection angles; and a third generating unit configured to two-dimensionally arrange the plurality of pieces of projection data based on the plurality of projection angles, to thereby generate a two-dimensional image.

The present invention also encompasses an image processing method to be performed by the image processing apparatus described above, and a computer readable medium.

Further features of the present invention will become apparent from the following description of exemplary embodiments with reference to the attached drawings.

### BRIEF DESCRIPTION OF THE DRAWINGS

FIG. 1A is a diagram for illustrating an example of a schematic configuration of a radiation imaging system in a first embodiment of the present invention.

FIG. 1B is a diagram for illustrating an example of the schematic configuration of the radiation imaging system in the first embodiment of the present invention.

FIG. 2A is a diagram for illustrating a display example of a diagnostic image.

FIG. 2B is a diagram for illustrating a display example of a diagnostic image.

FIG. 3A is a diagram for illustrating a processing example of a projection data generating unit and a two-dimensional data generating unit of FIG. 1B in the first embodiment of the present invention.

FIG. 3B is a diagram for illustrating a processing example of the projection data generating unit and the two-dimensional data generating unit of FIG. 1B in the first embodiment of the present invention.

FIG. 3C is a diagram for illustrating a processing example of the projection data generating unit and the two-dimensional data generating unit of FIG. 1B in the first embodiment of the present invention.

FIG. 4A is a diagram for illustrating a processing example of the projection data generating unit and the two-dimensional data generating unit of FIG. 1B obtained when a breast is applied as an imaging target object in the first embodiment of the present invention.

FIG. 4B is a diagram for illustrating a processing example of the projection data generating unit and the two-dimensional data generating unit of FIG. 1B obtained when the breast is applied as the imaging target object in the first embodiment of the present invention.

FIG. 4C is a diagram for illustrating a processing example of the projection data generating unit and the two-dimensional data generating unit of FIG. 1B obtained when the breast is applied as the imaging target object in the first embodiment of the present invention.

FIG. 5A is a diagram for illustrating a processing example of the projection data generating unit and the two-dimensional data generating unit of FIG. 1B in a second embodiment of the present invention.

FIG. 5B is a diagram for illustrating a processing example of the projection data generating unit and the two-dimensional data generating unit of FIG. 1B in the second embodiment of the present invention.

FIG. 5C is a diagram for illustrating a processing example of the projection data generating unit and the two-dimensional data generating unit of FIG. 1B in the second embodiment of the present invention.

FIG. 6A is a diagram for illustrating a processing example of the projection data generating unit and the two-dimensional data generating unit of FIG. 1B obtained when a breast is applied as an imaging target object in the second embodiment of the present invention.

FIG. 6B is a diagram for illustrating a processing example of the projection data generating unit and the two-dimensional data generating unit of FIG. 1B obtained when the breast is applied as the imaging target object in the second embodiment of the present invention.

FIG. 6C is a diagram for illustrating a processing example of the projection data generating unit and the two-dimensional data generating unit of FIG. 1B obtained when the breast is applied as the imaging target object in the second embodiment of the present invention.

FIG. 6D is a diagram for illustrating a processing example of the projection data generating unit and the two-dimensional data generating unit of FIG. 1B obtained when the breast is applied as the imaging target object in the second embodiment of the present invention.

FIG. 7A is a diagram for illustrating a processing example of the projection data generating unit and the two-dimensional data generating unit of FIG. 1B in a third embodiment of the present invention.

FIG. 7B is a diagram for illustrating a processing example of the projection data generating unit and the two-dimensional data generating unit of FIG. 1B in the third embodiment of the present invention.

FIG. 7C is a diagram for illustrating a processing example of the projection data generating unit and the two-dimensional data generating unit of FIG. 1B in the third embodiment of the present invention.

FIG. 8A is a diagram for illustrating a display example of a diagnostic image in a fourth embodiment of the present invention.

FIG. 8B is a diagram for illustrating a display example of a diagnostic image in the fourth embodiment of the present invention.

FIG. 9 is a diagram for illustrating axial, coronal, and sagittal sections of an imaging target object.

### DESCRIPTION OF THE EMBODIMENTS

Now, modes for carrying out the present invention (embodiments) are described with reference to the drawings.

### First Embodiment

First, a first embodiment of the present invention is described.

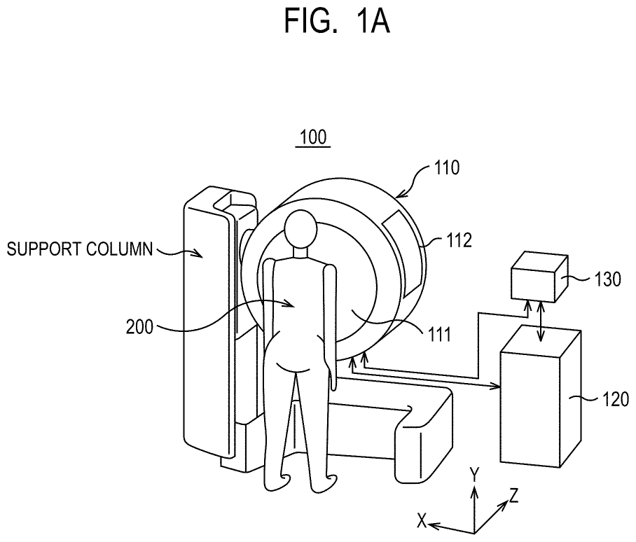

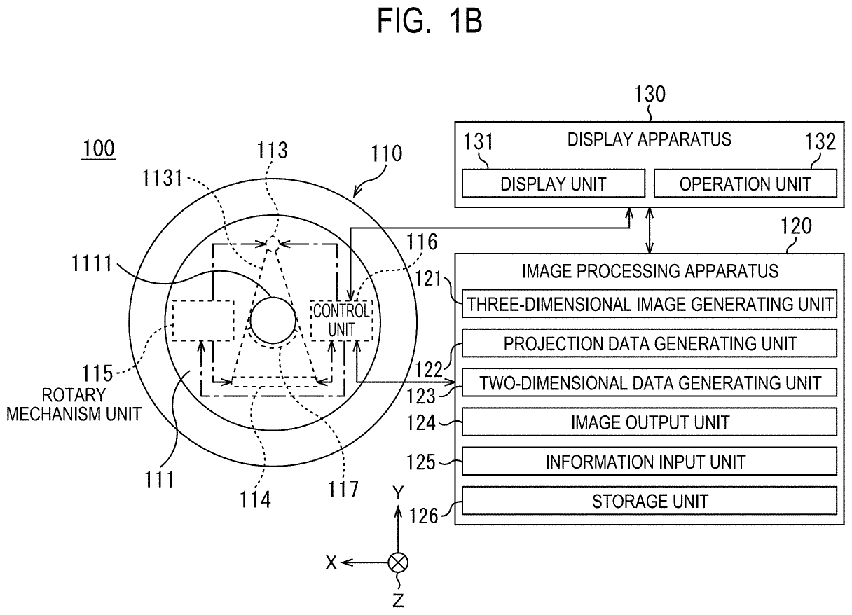

FIG. 1A and FIG. 1B are diagrams for illustrating examples of a schematic configuration of a radiation imaging system 100 in the first embodiment of the present invention. In the first embodiment, an example of applying, as the radiation imaging system 100, a system configured to perform what is called computed tomography (CT), in which an imaging target object is scanned with a radiation to compose an internal image of the imaging target object through image processing by a computer is described. Further, in the first embodiment, an example of applying, as the imaging target object, a breast of a subject 200 to be examined (hereinafter referred to as “subject 200) is described.

As illustrated in FIG. 1A, the radiation imaging system 100 includes a radiation imaging apparatus 110, an image processing apparatus 120, and a display device 130.

As illustrated in FIG. 1A, the radiation imaging apparatus 110 is mounted on a support column to be ascendable/descendable and tiltable. Further, as illustrated in FIG. 1A, the radiation imaging apparatus 110 includes, on the outside thereof, a support plate 111 and an access window 112. The support plate 111 has an opening portion (opening portion 1111 illustrated in FIG. 1B) for inserting the breast, which is the imaging target object, and is a plate-shaped member to be brought into the subject 200 when the subject 200 inserts her breast into the opening portion. Further, the access window 112 is configured to be capable of being opened/closed, and when this access window 112 is opened, an examiner, for example, an imaging operator, can check whether the breast inserted from the opening portion of the support plate 111 is arranged in an imaging region. Further, the access window 112 is closed during radiation imaging. FIG. 1A also shows an XYZ coordinate system in which a direction in which the subject 200 inserts the breast into the opening portion of the support plate 111 is defined as a Z direction, a direction from the legs to the head of the subject 200 when the subject 200 is in a standing position is defined as a Y direction, and a direction perpendicular to the Z direction and the Y direction is defined as an X direction. In the example illustrated in FIG. 1A, a form in which radiation imaging is performed under a state in which the subject 200 is in the standing position is illustrated. However, the present invention is not limited to this form, and is also applicable to a form in which for example, radiation imaging is performed under a state in which the subject 200 is in a prone position.

FIG. 1B is a diagram for illustrating an example of an internal configuration of the radiation imaging apparatus 110 illustrated in FIG. 1A. Also in FIG. 1B, an XYZ coordinate system similar to that of FIG. 1A is illustrated. Specifically, in FIG. 1B, components located inside the support plate 111 of the radiation imaging apparatus 110 are illustrated with dotted lines.

As illustrated in FIG. 1B, in the support plate 111, the opening portion 1111 for inserting the breast, which is the imaging target object, is formed. Further, the radiation imaging apparatus 110 includes, in addition to the support plate 111 and the access window 112 illustrated in FIG. 1A, a radiation generating unit 113, a radiation detection unit 114, a rotary mechanism unit 115, a control unit 116, and a breast holding unit 117 as illustrated in FIG. 1B.

The radiation generating unit 113 is configured to generate a radiation 1131, for example, an X-ray, to the breast of the subject 200 inserted from the opening portion 1111 of the support plate 111. Specifically, in the first embodiment, the radiation generating unit 113 is configured to output the radiation 1131, which is a quadrangular cone beam, for example, to the breast of the subject 200 inserted from the opening portion 1111 and to the radiation detection unit 114.

The radiation detection unit 114 is arranged at a position opposed to the radiation generating unit 113, and is configured to detect the incident radiation 1131 as an image signal, which is an electrical signal. When the breast of the subject 200 is imaged with radiation, the radiation detection unit 114 is arranged at a position opposed to the radiation generating unit 113 with interposition of the breast of the subject 200 inserted from the opening portion 1111.

The rotary mechanism unit 115 is a mechanism unit configured to rotate the radiation generating unit 113 and the radiation detection unit 114 in a plane parallel to a surface (XY plane) of the support plate 111 with interposition of the breast of the subject 200 inserted from the opening portion 1111. Here, in the first embodiment, the rotary mechanism unit 115 is configured to have an axis of rotation at the center of the opening portion 1111. Further, for example, when the breast of the subject 200 is imaged with radiation (imaged by CT), the rotary mechanism unit 115 rotates the radiation generating unit 113 and the radiation detection unit 114 by 360° around the breast.

The control unit 116 is configured to control the components of the radiation imaging apparatus 110 for centralized control of operation of the radiation imaging apparatus 110, and to perform various kinds of processing. When the breast of the subject 200 is imaged by CT, the control unit 116 controls a timing to generate the radiation 1131 from the radiation generating unit 113, controls a timing to detect the radiation 1131 by the radiation detection unit 114, and controls the rotation by the rotary mechanism unit 115, for example. The control unit 116 further performs processing of transmitting the image signal obtained by detecting the radiation 1131 by the radiation detection unit 114 to the image processing apparatus 120, for example.

The breast holding unit 117 is a mechanism unit configured to hold the breast so that the breast of the subject 200 inserted from the opening portion 1111 of the support plate 111 fits into a radiation imaging region. The breast holding unit 117 is formed of a material that transmits the radiation 1131.

The image processing apparatus 120 is configured to process the image signal obtained from the radiation imaging apparatus 110 through the detection by the radiation detection unit 114. As illustrated in FIG. 1B, the image processing apparatus 120 includes a three-dimensional image generating unit 121, a projection data generating unit 122, a two-dimensional data generating unit 123, an image output unit 124, an information input unit 125, and a storage unit 126.

The three-dimensional image generating unit 121 is configured to receive the image signal obtained by imaging the breast of the subject 200, which is the imaging target object, with the use of the radiation 1131 in the radiation imaging apparatus 110, and reconstruct and generate a three-dimensional image with the use of the image signal. The three-dimensional image generating unit 121 is a component corresponding to an example of a “first generating unit” in at least one embodiment of the present invention.

The projection data generating unit 122 is configured to set a projection reference line in the three-dimensional image generated by the three-dimensional image generating unit 121. Then, the projection data generating unit 122 performs projection processing of projecting pixel values of the three-dimensional image to the projection reference line for each projection angle of a plurality of projection angles about the set projection reference line to generate a plurality of pieces of projection data at the plurality of projection angles. At this time, the projection data generating unit 122 may perform, as the projection processing described above, processing of calculating a sum of the pixel values of the three-dimensional image, processing of calculating an average of the pixel values of the three-dimensional image, or processing of calculating a maximum value of the pixel values of the three-dimensional image. The projection data generating unit 122 is a component corresponding to an example of a “second generating unit” in at least one embodiment of the present invention.

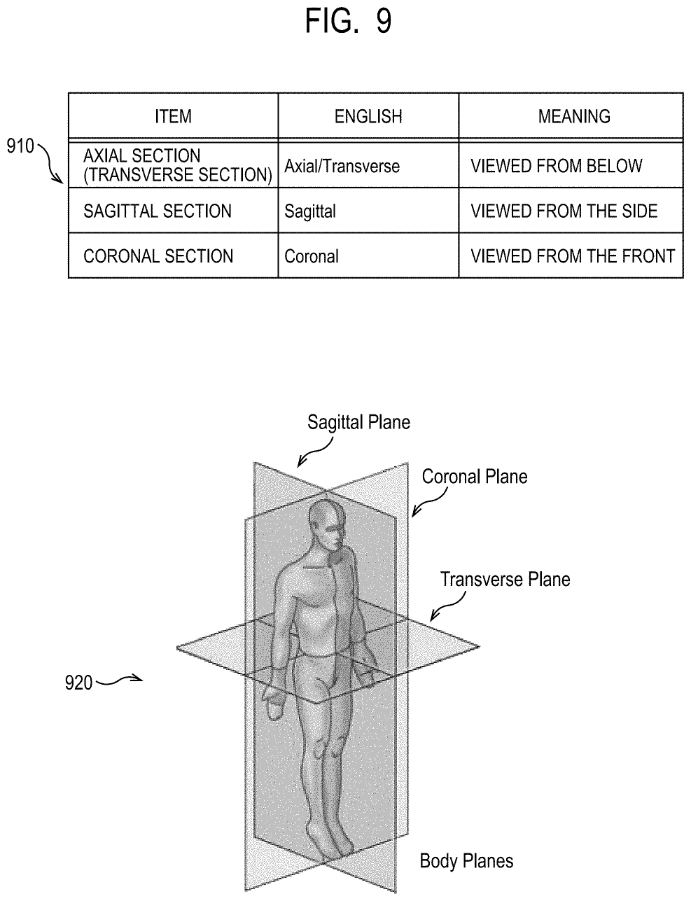

The two-dimensional data generating unit 123 is configured to two-dimensionally arrange the plurality of pieces of projection data based on the plurality of projection angles obtained through the processing by the projection data generating unit 122, to thereby generate a radial image, which is a two-dimensional image. The two-dimensional data generating unit 123 may further generate, in the three-dimensional image generated by the three-dimensional image generating unit 121, at least one standard image selected from an axial image based on an axial section illustrated in FIG. 9, a coronal image based on a coronal section illustrated in FIG. 9, and a sagittal image based on a sagittal section illustrated in FIG. 9. In the first embodiment, the two-dimensional data generating unit 123 generates all of the following standard images: the axial image, the coronal image, and the sagittal image. The two-dimensional data generating unit 123 is a component corresponding to an example of a “third generating unit” in at least one embodiment of the present invention.

The image output unit 124 is configured to output the two-dimensional image generated by the two-dimensional data generating unit 123 to the display device 130. The image output unit 124 is also configured to output the three-dimensional image generated by the three-dimensional image generating unit 121, and the projection data generated by the projection data generating unit 122 to the display device 130 as required.

The information input unit 125 is configured to input various kinds of information to the image processing apparatus 120.

The storage unit 126 is configured to store a program for controlling operation of the image processing apparatus 120, and various kinds of information and various kinds of data required when the operation of the image processing apparatus 120 is performed. The storage unit 126 is also configured to store various kinds of information and various kinds of data obtained based on the operation of the image processing apparatus 120. For example, in the storage unit 126, the three-dimensional image generated by the three-dimensional image generating unit 121, the projection data generated by the projection data generating unit 122, and the two-dimensional image generated by the two-dimensional data generating unit 123 are also stored.

The display device 130 is configured to display, for example, various images and various kinds of information to the examiner, for example, an imaging operator. As illustrated in FIG. 1B, the display device 130 includes a display unit 131 and an operation unit 132. The display unit 131 is a component configured to display various images and various kinds of information to the examiner, and the operation unit 132 is a component configured to receive input of an operation by the examiner.

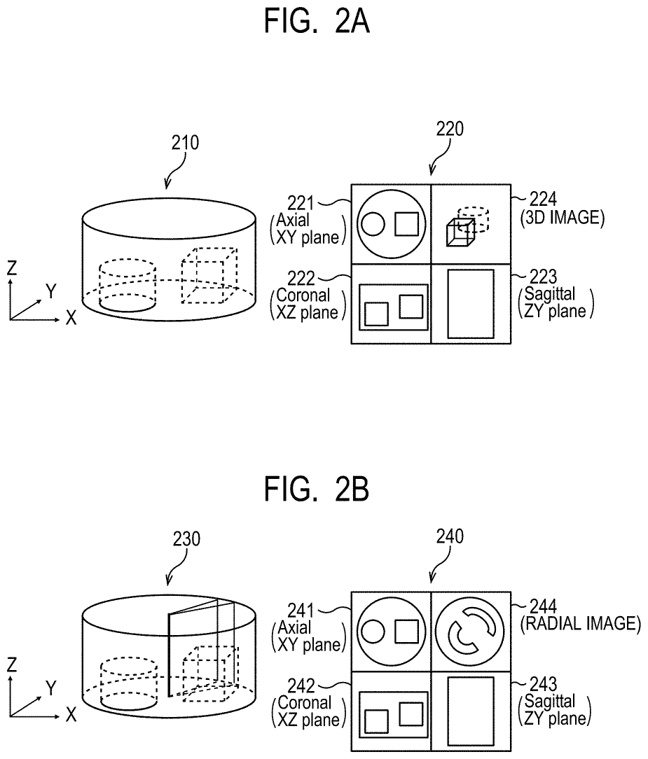

FIG. 2A and FIG. 2B are diagrams for illustrating display examples of a diagnostic image. FIG. 2A is a diagram for illustrating a display example of a diagnostic image in a comparative example, and FIG. 2B is a diagram for illustrating a display example of a diagnostic image in the first embodiment of the present invention. In FIG. 2A and FIG. 2B, XYZ coordinate systems similar to those of FIG. 1A and FIG. 1B are illustrated.

In the comparative example illustrated in FIG. 2A, on a display screen 220, standard images of an axial image 221, a coronal image 222, and a sagittal image 223 in a three-dimensional image 210 illustrated in FIG. 2A, and a three-dimensional image 224, for example, a volume rendering (VR) image, are displayed in 2×2, that is, 4 parts.

In the first embodiment of the present invention illustrated in FIG. 2B, on a display screen 240 of the display unit 131, standard images of an axial image 241, a coronal image 242, and a sagittal image 243 in a three-dimensional image 230 illustrated in FIG. 2B, and a radial image 244 are displayed in 2×2, that is, 4 parts. In at least one embodiment of present invention, the radial image 244 may be displayed alone, but when the radial image 244 is displayed along with the other standard images (241 to 243) as illustrated in FIG. 2B, there is an advantage in that a relationship with a standard section in the imaging target object can be checked. In this case, in the first embodiment, there is adopted a form in which the two-dimensional data generating unit 123 generates the standard images of the axial image 241, the coronal image 242, and the sagittal image 243. Further, in the first embodiment, there is adopted a form in which the image output unit 124 outputs, along with the standard images, the radial image 244 in association with the standard images to the display unit 131.

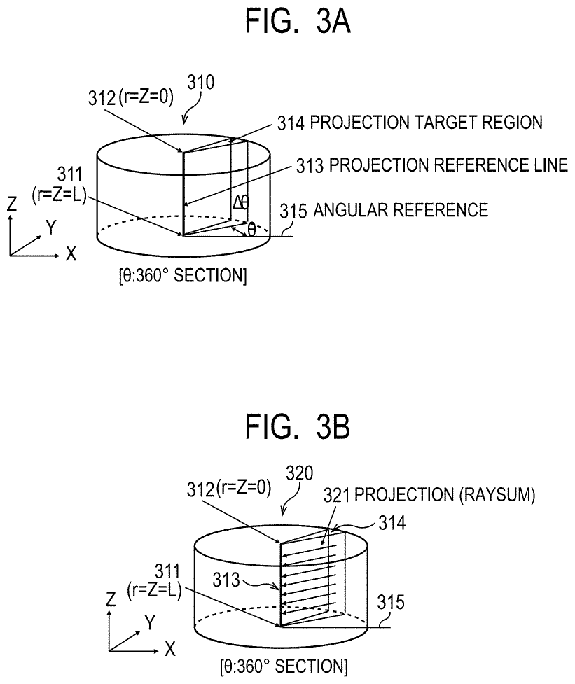

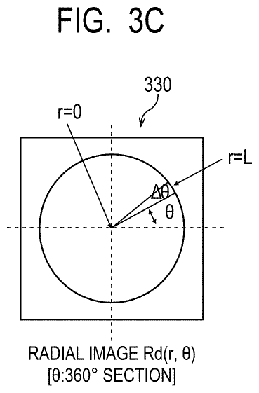

FIG. 3A to FIG. 3C are diagrams for illustrating processing examples of the projection data generating unit 122 and the two-dimensional data generating unit 123 of FIG. 1B in the first embodiment of the present invention. In FIG. 3A and FIG. 3B, XYZ coordinate systems similar to those of FIG. 1A and FIG. 1B are also illustrated.

FIG. 3A shows a three-dimensional image 310 generated by the three-dimensional image generating unit 121. Then, the projection data generating unit 122 sets a projection reference line 313 in the three-dimensional image illustrated in FIG. 3A. For example, the projection data generating unit 122 sets, as the projection reference line 313, a line connecting a point 311 and a point 312, which are input through operations to the operation unit 132 by the examiner, and input via the information input unit 125. In this example, the point 311 is at a position of r=Z=L, and the point 312 is at a position of r=Z=0. In FIG. 3A and FIG. 3B, for simpler description, an example in which the projection reference line 313 is set in a direction parallel to the Z direction is illustrated. However, the present invention is not limited to this form, and the projection reference line 313 may be set at any position and any angle in an XYZ space in the three-dimensional image. Further, FIG. 3A shows an example of a projection target region 314, and an example of an angular reference 315 of the 360° perimeter about the projection reference line 313.

The two-dimensional data generating unit 123 in the first embodiment can generate, through setting the projection reference line 313 by the projection data generating unit 122, a radial image (radial image 330 illustrated in FIG. 3C) from the 360° perimeter direction about the projection reference line 313. Here, consideration is made of a projection target region G(θ, Δθ), which is the projection target region 314 in a AO section at a position rotated by an angle θ from the angular reference 315 in the three-dimensional image 310. At this time, as illustrated in FIG. 3A, the projection target region G(θ, Δθ) is a column having an arc section and a height L.

FIG. 3B shows a three-dimensional image 320 similar to the three-dimensional image 310 illustrated in FIG. 3A. Further, FIG. 3B shows projection processing 321 in which pixel values of the three-dimensional image 320 included in the projection target region G(θ, Δθ) are projected to the projection reference line 313 by the projection data generating unit 122. At this time, the projection data generating unit 122 performs the projection processing 321 so that the pixel values of the three-dimensional image 320 included in the projection target region G(θ, Δθ) are projected substantially perpendicularly to the projection reference line 313 for each projection angle, to thereby generate a plurality of pieces of projection data at a plurality of projection angles. At this time, in one cycle of the projection processing 321 based on a projection angle, one piece of one-dimensional projection data is generated. For example, when projection data P(r, θ, Δθ) is calculated with the angle θ being 0° to 360° and with AO being 1°, 360 lines of projection data P are generated.

In the example illustrated in FIG. 3B, an example in which, as the projection processing 321, the processing (RAYSUM) of calculating the sum of the pixel values of the three-dimensional image is illustrated, but in at least one embodiment of the present invention, the processing of calculating the average of the pixel values of the three-dimensional image, or the processing of calculating the maximum value of the pixel values of the three-dimensional image may be adopted instead. For example, when a lesion of low contrast is to be viewed, the processing (RAYSUM) of calculating the sum of the pixel values of the three-dimensional image is suitably performed as the projection processing 321. Meanwhile, for example, when a lesion of high contrast is to be viewed, the processing of calculating the maximum value of the pixel values of the three-dimensional image is suitably performed as the projection processing 321.

FIG. 3C shows the radial image 330, which is a two-dimensional image obtained by arranging, by the two-dimensional data generating unit 123, the plurality of pieces of projection data P(r, θ, Δθ) described above on the arc based on the projection angle. Here, the radial image 330 can also be regarded as a two-dimensional image obtained by arranging the plurality of pieces of projection data P(r, θ, Δθ) on polar coordinates based on the projection angle.

Now, one piece of projection data at the projection angle of the angle θ from the angular reference 315 illustrated in FIG. 3A is described, for example. In this case, there is obtained a form in which, in the radial image 330 illustrated in FIG. 3C, data of the point 312 of the one piece of projection data is arranged as data of r=0 illustrated in FIG. 3C, data of the point 311 is arranged as data of r=L at the angle θ illustrated in FIG. 3C, and pieces of data at the other points are arranged between those pieces of data. The other pieces of projection data are also arranged in accordance with the projection angle similarly to the projection angle described here.

For example, the radial image 330 is a square image (for example, 1,024 pixels×1,024 pixels), and hence interpolation calculation is required in arranging the projection data P(r, θ, Δθ). Specifically, the calculation is performed by linear interpolation between projection data P(r, θ−1, Δθ) and projection data P(r, θ+1, Δθ). It should be noted, however, that the projection data, which is one-dimensional data, is mapped on the arc, and hence a resolution is reduced as “r” becomes larger. In this case, in the first embodiment, the resolution can be increased through reducing an angle pitch from an angle of 0° to an angle of 360°. Further, it is not required that the angle pitch be constant from the angle of 0° to the angle of 360°, and the angle pitch may be increased only for a region without a lesion in order to reduce computational complexity.

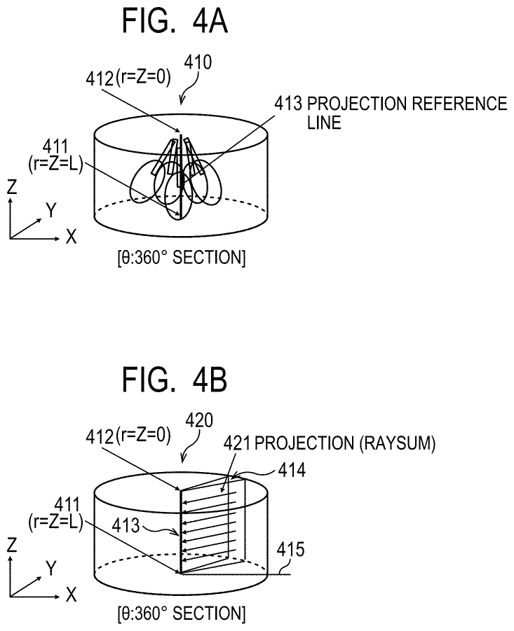

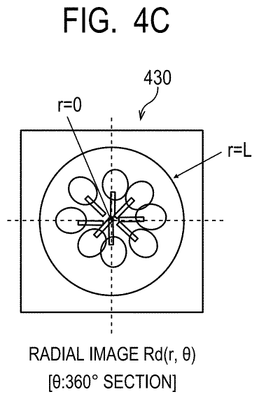

FIG. 4A to FIG. 4C are diagrams for illustrating processing examples of the projection data generating unit 122 and the two-dimensional data generating unit 123 of FIG. 1B obtained when the breast is applied as the imaging target object in the first embodiment of the present invention. Further, FIG. 4A and FIG. 4B show XYZ coordinate systems similar to those of FIG. 1A and FIG. 1B. In the following description of FIG. 4A to FIG. 4C, matters common to the description of FIG. 3A to FIG. 3C described above are omitted as required.

FIG. 4A shows a three-dimensional image 410 generated by the three-dimensional image generating unit 121. In the three-dimensional image 410, there are a plurality of sets of lactiferous ducts indicated by rectangles and lobes of mammary gland indicated by ellipses, and a portion in which the lactiferous ducts indicated by the rectangles gather is a papilla. The projection data generating unit 122 sets a projection reference line 413 in the three-dimensional image illustrated in FIG. 4A. For example, the projection data generating unit 122 sets, as the projection reference line 413, a line connecting a point 411 and a point 412, which are input through operations to the operation unit 132 by the examiner, and input via the information input unit 125. In the example illustrated in FIG. 4A, the projection reference line 413 is set vertically (in the Z direction) from the papilla to a base (XY plane) of the three-dimensional image.

FIG. 4B shows a three-dimensional image 420 similar to the three-dimensional image 410 illustrated in FIG. 4A. Further, FIG. 4B shows projection processing 421 in which pixel values of the three-dimensional image 420 included in a projection target region 414 are projected to the projection reference line 413 by the projection data generating unit 122. At this time, the projection data generating unit 122 performs the projection processing 421 so that the pixel values of the three-dimensional image 420 are projected substantially perpendicularly to the projection reference line 413 for each projection angle, to thereby generate a plurality of pieces of projection data at a plurality of projection angles. Further, FIG. 4B shows an example of an angular reference 415 to serve as a reference of the projection angles.

FIG. 4C shows a radial image 430, which is a two-dimensional image obtained by arranging, by the two-dimensional data generating unit 123, the plurality of pieces of projection data generated by the projection data generating unit 122 on an arc based on the projection angle. The radial image 430 allows the sets of the lactiferous ducts indicated by the rectangles and the lobes of mammary gland indicated by the ellipses to be observed in a developed view to facilitate determination as to whether the structure of the lesion is regional or segmental, and is a two-dimensional image suitable as a diagnostic image for the breast filled with tissues. The radial image 430 is a two-dimensional image obtained by arranging the lobes of mammary gland indicated by the ellipses on the arc around the papilla, and hence facilitates determination as to whether the lesion is segmental. Further, for example, the radial image 430 can be used as an image that facilitates determination as to whether the lesion (microcalcification and tumor) is diffused, regional, grouped, or segmental by focusing attention on the arrangement of mammary glands. Here, observation as to whether the lesion is diffused, regional, grouped, or segmental is important in, for example, diagnosing whether the lesion is malign/benign because breast cancer has a characteristic of growing along mammary grand tissues.

### Second Embodiment

Next, a second embodiment of the present invention is described. In the following description of the second embodiment, description of matters common to the first embodiment described above is omitted, and matters different from the first embodiment described above are described.

A schematic configuration of a radiation imaging system in the second embodiment is similar to the schematic configuration of the radiation imaging system 100 in the first embodiment illustrated in FIG. 1A and FIG. 1B. Specifically, a schematic configuration of the image processing apparatus according to the second embodiment is similar to the schematic configuration of the image processing apparatus 120 according to the first embodiment illustrated in FIG. 1A and FIG. 1B.

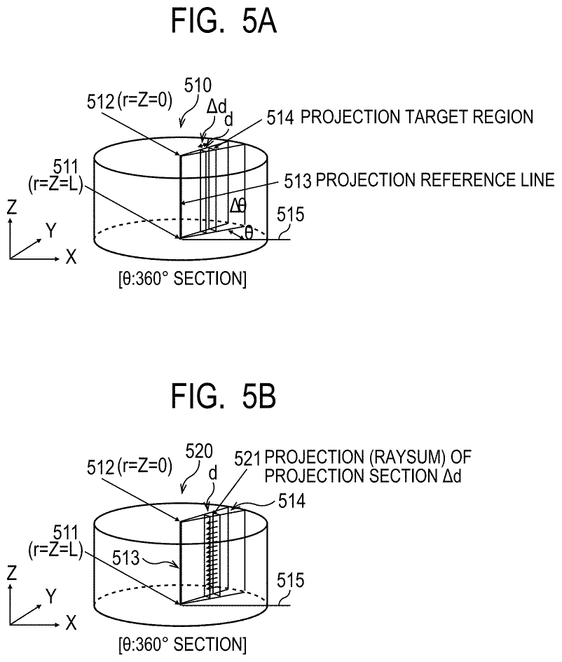

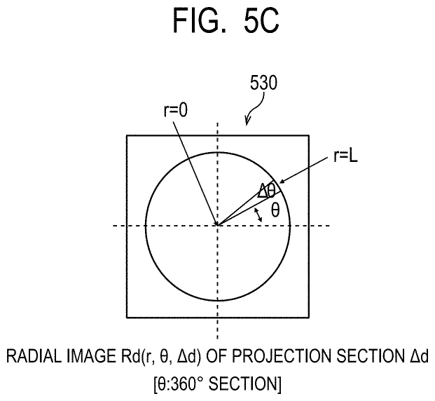

FIG. 5A to FIG. 5C are diagrams for illustrating processing examples of the projection data generating unit 122 and the two-dimensional data generating unit 123 of FIG. 1B in the second embodiment of the present invention. In FIG. 5A and FIG. 5B, XYZ coordinate systems similar to those of FIG. 1A and FIG. 1B are also illustrated. Here, a difference between the second embodiment and the first embodiment is that, in the second embodiment, a projection section to the projection reference line is divided. Specifically, in the first embodiment, a projection section of the projection target region is from the projection reference line to the periphery of the three-dimensional image, but in the second embodiment, the projection section is divided to perform processing for each projection section Δd (see FIG. 5A). Through adjustment of a size of the projection section Δd, the projection target region can be reduced to generate a more detailed radial image. At this time, a position and the size of the projection section Δd can be set freely by, for example, an input through an operation to the operation unit 132.

FIG. 5A shows a three-dimensional image 510 generated by the three-dimensional image generating unit 121. Then, the projection data generating unit 122 sets a projection reference line 513 in the three-dimensional image illustrated in FIG. 5A. For example, the projection data generating unit 122 sets, as the projection reference line 513, a line connecting a point 511 and a point 512, which are input through operations to the operation unit 132 by the examiner, and input via the information input unit 125. FIG. 5A also shows an example of a projection target region 514, an example of an angular reference 515 of the 360° perimeter about the projection reference line 513, and an example of the projection section Δd described above.

Specifically, in the second embodiment, the projection data generating unit 122 divides a three-dimensional image 510 into a plurality of projection sections Δd depending on a distance to the projection reference line 513 based on the input through the operation to the operation unit 132. Then, the projection data generating unit 122 adopts a form of generating a plurality of pieces of projection data for each projection section of the plurality of projection sections obtained by the division. Further, in the second embodiment, the two-dimensional data generating unit 123 adopts a form of generating a radial image for each projection section of the plurality of projection sections. Now, description is made with reference to FIG. 5B and FIG. 5C.

FIG. 5B shows a three-dimensional image 520 similar to the three-dimensional image 510 illustrated in FIG. 5A. Further, FIG. 5B shows projection processing 521 in which pixel values of the three-dimensional image 520 included in a projection target region G(θ, Δθ, d, Δd), which is the projection target region 514, are projected to the projection reference line 513 by the projection data generating unit 122. At this time, the projection data generating unit 122 performs the projection processing 521 so that the pixel values of the three-dimensional image 520 included in the projection target region G(θ, Δθ, d, Δd) are projected substantially perpendicularly to the projection reference line 513 for each projection section and for each projection angle to generate the plurality of pieces of projection data.

In the example illustrated in FIG. 5B, an example in which, as the projection processing 521, the processing (RAYSUM) of calculating the sum of the pixel values of the three-dimensional image is illustrated, but in at least one embodiment of the present invention, the processing of calculating the average of the pixel values of the three-dimensional image, or the processing of calculating the maximum value of the pixel values of the three-dimensional image may be adopted instead.

FIG. 5C shows a radial image 530, which is a two-dimensional image obtained by arranging, by the two-dimensional data generating unit 123, the plurality of pieces of projection data in one projection section (d, Δd) on an arc based on the projection angle. Here, the radial image 530 can also be regarded as a two-dimensional image obtained by arranging the plurality of pieces of projection data on polar coordinates based on the projection angle while changing “d” and Δd in the one projection section (d, Δd) individually or at the same time.

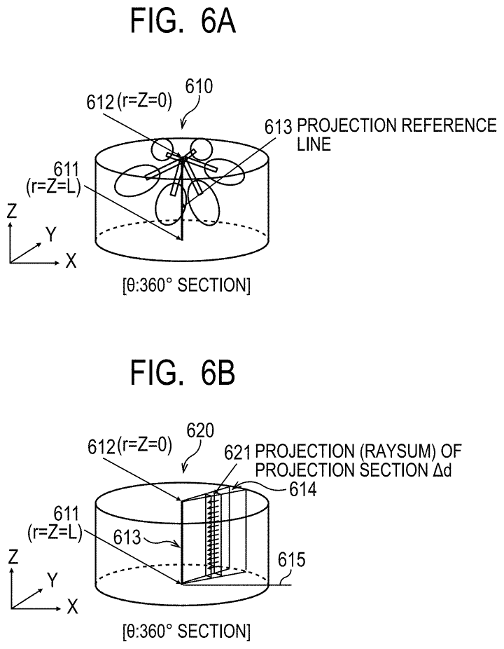

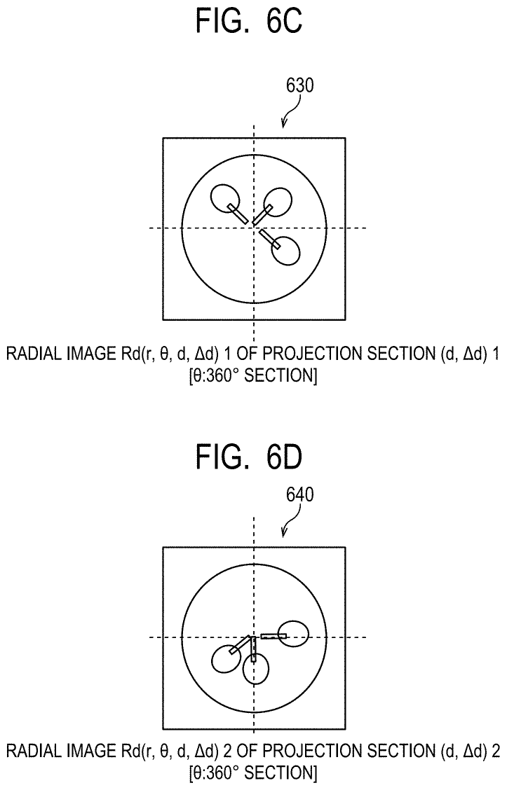

FIG. 6A to FIG. 6D are diagrams for illustrating processing examples of the projection data generating unit 122 and the two-dimensional data generating unit 123 of FIG. 1B obtained when the breast is applied as the imaging target object in the second embodiment of the present invention. Further, FIG. 6A and FIG. 6B show XYZ coordinate systems similar to those of FIG. 1A and FIG. 1B. In the following description of FIG. 6A to FIG. 6D, matters common to the description of FIG. 5A to FIG. 5C described above are omitted as required.

FIG. 6A shows a three-dimensional image 610 generated by the three-dimensional image generating unit 121. In the three-dimensional image 610, there are a plurality of sets of lactiferous ducts indicated by rectangles and lobes of mammary gland indicated by ellipses, and a portion in which the lactiferous ducts indicated by the rectangles gather is a papilla. The three-dimensional image 610 illustrated in FIG. 6A is different from the three-dimensional image 410 illustrated in FIG. 4A in that some of the lobes of mammary gland indicated by the ellipses are at positions close to a center position (position of a projection reference line 613) of the three-dimensional image, and others are at positions far from the center position.

The projection data generating unit 122 sets a projection reference line 613 in the three-dimensional image illustrated in FIG. 6A. For example, the projection data generating unit 122 sets, as the projection reference line 613, a line connecting a point 611 and a point 612, which are input through operations to the operation unit 132 by the examiner, and input via the information input unit 125. In the example illustrated in FIG. 6A, the projection reference line 613 is set vertically (in the Z direction) from the papilla to a base (XY plane) of the three-dimensional image.

FIG. 6B shows a three-dimensional image 620 corresponding to the three-dimensional image 610 illustrated in FIG. 6A. Further, FIG. 6B shows projection processing 621 in which pixel values of the three-dimensional image 620 included in a projection section (d, Δd) of a projection target region 614 are projected to the projection reference line 613 by the projection data generating unit 122. At this time, the projection data generating unit 122 performs the projection processing 621 so that the pixel values of the three-dimensional image 620 are projected substantially perpendicularly to the projection reference line 613 for each projection section and for each projection angle to generate the plurality of pieces of projection data. Further, FIG. 6B shows an example of an angular reference 615 to serve as a reference of the projection angles.

FIG. 6C shows a radial image 630, which is a two-dimensional image obtained by arranging, by the two-dimensional data generating unit 123, a plurality of pieces of projection data in a projection section (d, Δd) 1 that is far from the position of the projection reference line 613, which are generated by the projection data generating unit 122, on an arc based on the projection angle.

FIG. 6D shows a radial image 640, which is a two-dimensional image obtained by arranging, by the two-dimensional data generating unit 123, a plurality of pieces of projection data in a projection section (d, Δd) 2 that is close from the position of the projection reference line 613, which are generated by the projection data generating unit 122, on an arc based on the projection angle.

In the examples illustrated in FIG. 6C and FIG. 6D, different sets of lactiferous ducts and lobes of mammary gland can be observed in the radial image 630 of the projection section (d, Δd) 1 and the radial image 640 of the projection section (d, Δd) 2. As a result, in the second embodiment, a radial image with which diagnosis as to whether the lesion is regional or segmental can be performed in more detail than in the first embodiment can be provided. Further, through adjustment of the projection section (d, Δd) to the projection reference line 613, overlapping of the mammary glands can be further removed to facilitate diagnosis as to whether the lesion is regional, grouped, or segmental.

Specifically, in the second embodiment, through setting a plurality of projection sections (d, Δd), the two-dimensional data generating unit 123 generates a plurality of radial images. Then, in the case of the second embodiment, the image output unit 124 may take a form in which the radial images for each projection section (d, Δd) generated by the two-dimensional data generating unit 123 are collectively output to the display device 130, or a form in which the radial images are output to the display device 130 in a switching manner for each projection section (d, Δd). As a result, the display device 130 may take a form in which the plurality of radial images for each projection section (d, Δd) are displayed collectively on a display screen of the display unit 131, or a form in which the radial images are displayed on the display screen of the display unit 131 in a switching manner for each projection section (d, Δd). Here, in the case of the form in which the radial images are displayed on the display screen of the display unit 131 in a switching manner for each projection section (d, Δd), for example, there may be adopted a form in which, through changing the size and the position of the projection section (d, Δd), the radial images are displayed in a manner similar to a moving image in real time as the radial image 244 on the display screen 240 illustrated in FIG. 2B.

### Third Embodiment

Next, a third embodiment of the present invention is described. In the following description of the third embodiment, description of matters common to the first embodiment described above is omitted, and matters different from the first embodiment described above are described.

A schematic configuration of a radiation imaging system in the third embodiment is similar to the schematic configuration of the radiation imaging system 100 in the first embodiment illustrated in FIG. 1A and FIG. 1B. Specifically, a schematic configuration of the image processing apparatus according to the third embodiment is similar to the schematic configuration of the image processing apparatus 120 according to the first embodiment illustrated in FIG. 1A and FIG. 1B.

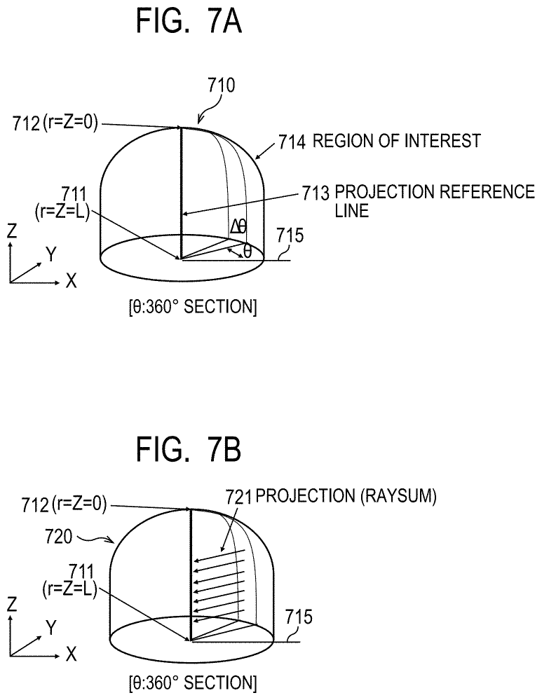

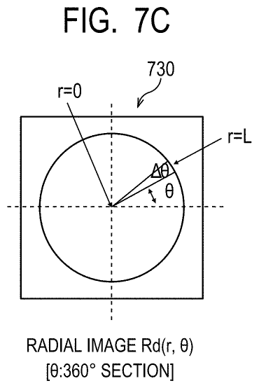

FIG. 7A to FIG. 7C are diagrams for illustrating processing examples of the projection data generating unit 122 and the two-dimensional data generating unit 123 of FIG. 1B in the third embodiment of the present invention. In FIG. 7A and FIG. 7B, XYZ coordinate systems similar to those of FIG. 1A and FIG. 1B are also illustrated. Here, a difference between the third embodiment and the first embodiment is that, in the third embodiment, a shape of a breast, which is an imaging target object, is extracted as a region of interest from a three-dimensional image, and a projection reference line is set in the region of interest to perform processing. In other words, in the first embodiment, the projection section of the projection target region is from the projection reference line to the periphery of the three-dimensional image, but in the third embodiment, a projection section of the projection target region is from the projection reference line to the periphery of the region of interest.

FIG. 7A shows a region of interest 710 extracted, by the projection data generating unit 122, as the shape of the breast, which is the imaging target object, from a three-dimensional image generated by the three-dimensional image generating unit 121. Here, the projection data generating unit 122 may take a form in which, for example, the three-dimensional image is subjected to image processing to extract the shape of the breast, which is the imaging target object, as the region of interest 710 based on a predetermined shape, for example a cone, or any shape input through an operation to the operation unit 132. At this time, in the case of the form in which the region of interest 710 is extracted through subjecting of the three-dimensional image to the image processing, a threshold processing with the use of a binarization threshold value, region growing, or other image processing may be used.

Then, the projection data generating unit 122 sets a projection reference line 713 in the region of interest illustrated in FIG. 7A. For example, the projection data generating unit 122 sets, as the projection reference line 713, a line connecting a point 711 and a point 712, which are input through operations to the operation unit 132 by the examiner, and input via the information input unit 125. Further, FIG. 7A shows an example of an angular reference 715 of the 360° perimeter about the projection reference line 713. Further, when the shape of the breast is extracted as the region of interest 710, a position of the point 712 corresponds to a position of a papilla, and a base including the point 711 corresponds to a chest wall.

FIG. 7B shows projection processing 721 in which pixel values of the region of interest 710 included in the projection target region G(θ, Δθ) are projected to the projection reference line 713 by the projection data generating unit 122. At this time, the projection data generating unit 122 performs the projection processing 721 so that the pixel values of the region of interest 710 included in the projection target region G(θ, Δθ) are projected substantially perpendicularly to the projection reference line 713 for each projection angle, to thereby generate a plurality of pieces of projection data at a plurality of projection angles.

FIG. 7C shows a radial image 730, which is a two-dimensional image obtained by arranging, by the two-dimensional data generating unit 123, the plurality of pieces of projection data described above on the arc based on the projection angle.

In the third embodiment, through setting of the region of interest 710, the projection target region can be limited in advance. As a modification example of the third embodiment, there may be adopted a form in which the projection section (d, Δd) in the second embodiment is applied to the third embodiment described above to change a substantial thickness of a radial image.

In the third embodiment, through setting of the region of interest 710, the projection target region can be limited in advance. As a modification example of the third embodiment, in application of the projection section (d, Δd) in the second embodiment to the third embodiment described above, the distance from the projection reference line 713 to the periphery over which the projection processing 721 is performed to the projection reference line 713 is changed, and hence the projection section (d, Δd) may be set with a ratio depending on the above-mentioned periphery.

### Fourth Embodiment

Next, a fourth embodiment of the present invention is described. In the following description of the fourth embodiment, description of matters common to the first to third embodiments described above is omitted, and matters different from the first to third embodiments described above are described.

A schematic configuration of a radiation imaging system in the fourth embodiment is similar to the schematic configuration of the radiation imaging system 100 in the first embodiment illustrated in FIG. 1A and FIG. 1B. Specifically, a schematic configuration of the image processing apparatus according to the fourth embodiment is similar to the schematic configuration of the image processing apparatus 120 according to the first embodiment illustrated in FIG. 1A and FIG. 1B.

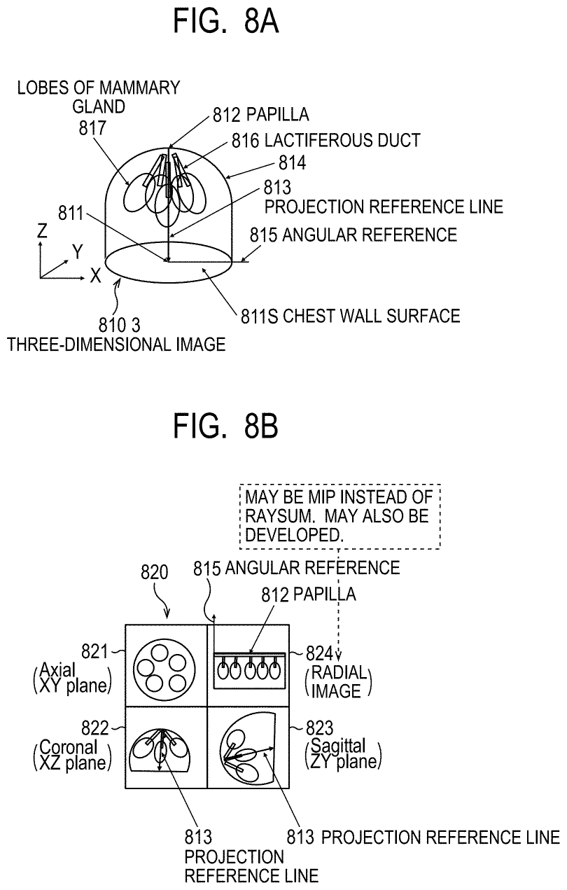

FIG. 8A and FIG. 8B are diagrams for illustrating display examples of diagnostic images in the fourth embodiment of the present invention. Further, in FIG. 8A, XYZ coordinate system similar to those of FIG. 1A and FIG. 1B are illustrated.

FIG. 8A shows a region of interest 814 extracted from a three-dimensional image 810, which corresponds to a region of interest 714 in the third embodiment illustrated in FIG. 7A. Further, in FIG. 8A, a chest wall 811 of a chest wall surface 811S in a breast is applied as a point corresponding to the point 711 in the third embodiment illustrated in FIG. 7A, and a papilla 812 in the breast is applied as a point corresponding to the point 712 in the third embodiment illustrated in FIG. 7A. In this case, the projection data generating unit 122 sets, as a projection reference line 813 illustrated in FIG. 8A, a line connecting the papilla 812 and the chest wall 811. FIG. 8A also shows an example of an angular reference 815 as a reference of the projection angles, and an example of lactiferous ducts 816 and lobes of mammary gland 817 located inside the breast.

Also in the fourth embodiment, the projection data generating unit 122 performs processing similar to that in the third embodiment.

Then, in the fourth embodiment, the two-dimensional data generating unit 123 generates a radial image (for example, radial image 824 illustrated in FIG. 8B) obtained by arranging the plurality of pieces of projection data generated by the projection data generating unit 122 in one direction in order of a plurality of projection angles.

In FIG. 8B, on a display screen 820 of the display unit 131, standard images of an axial image 821, a coronal image 822, and a sagittal image 823 in a three-dimensional image 810, and a radial image 824 are displayed in 2×2, that is, 4 parts. In this case, in the fourth embodiment, there is adopted a form in which the two-dimensional data generating unit 123 generates the standard images of the axial image 821, the coronal image 822, and the sagittal image 823. Further, in the fourth embodiment, there is adopted a form in which the image output unit 124 outputs, along with the standard images, the radial image 824 in association with the standard images to the display unit 131. Further, on the display screen 820, the projection reference line 813 is displayed in a changeable state on at least one standard image. Through changing of the projection reference line 813, in the fourth embodiment, the radial image 824 is sequentially regenerated and displayed. Through changing of the projection reference line 813, evaluation as to whether the lesion is regional or segmental can be performed in real time.

Further, in the fourth embodiment, there may be adopted a form in which, when any lobe of mammary gland 817 is selected on a standard image (in particular, coronal image 822) through the operation unit 132, a corresponding lobe of mammary gland 817 is highlighted on the radial image 824. Examples of the form of highlighting include highlighting a contour of the corresponding lobe of mammary gland 817, enclosing the corresponding lobe of mammary gland 817 with a region of interest (ROI), and changing a luminance of the corresponding lobe of mammary gland 817. Further, a part to be selected is not limited to the lobe of mammary gland 817, but may be microcalcification. The binarization threshold value used in the extraction is different for the lobes of mammary gland 817 and microcalcification, and hence there may be adopted a form in which the part to be highlighted is selected in advance through the operation unit 132 when being displayed in association. Further, in the fourth embodiment, the angular reference 815 may be changed to change a relationship between the lobe of mammary gland 817 at the left end and the lobe of mammary gland 817 at the right end illustrated in FIG. 8A in real time.

### Fifth Embodiment

Next, a fifth embodiment of the present invention is described. In the following description of the fifth embodiment, description of matters common to the fourth embodiment described above is omitted, and matters different from the fourth embodiment described above are described.

As the fifth embodiment, the projection section (d, Δd) in the second embodiment described above may be applied to the region of interest 814 in the fourth embodiment described above to change a substantial thickness of the radial image 824, and determine as to whether the lesion is regional or segmental in a manner similar to a moving image in real time.

### Other Embodiments

In the embodiments of the present invention described above, the examples assuming the CT image obtained by CT imaging are described. However, the present invention is not limited thereto, and an MM image obtained by MRI imaging and a PET image obtained by PET imaging are also applicable.

According to the embodiments described above, a two-dimensional image suitable as a diagnostic image can be generated even for an imaging target object filled with contents.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An image processing apparatus comprising:
a first generating unit configured to generate a three-dimensional image with use of an image signal obtained by imaging an imaging target object with a radiation;
a second generating unit configured to set a projection reference line in the three-dimensional image, and perform projection processing of projecting pixel values of the three-dimensional image to the projection reference line for each projection angle of a plurality of projection angles about the projection reference line, to thereby generate a plurality of pieces of projection data at the plurality of projection angles; and
a third generating unit configured to two-dimensionally arrange the plurality of pieces of projection data based on the plurality of projection angles, to thereby generate a two-dimensional image.

2. The image processing apparatus according to claim 1, wherein the second generating unit is configured to extract a shape of the imaging target object as a region of interest from the three-dimensional image, set the projection reference line in the region of interest, and project pixel values of the region of interest to the projection reference line for each projection angle, to thereby generate the plurality of pieces of projection data.

3. The image processing apparatus according to claim 2, wherein the second generating unit is configured to perform a threshold processing of the three-dimensional image, to thereby extract the shape of the imaging target object as the region of interest.

4. The image processing apparatus according to claim 1,
wherein the second generating unit is configured to divide the three-dimensional image into a plurality of projection sections in accordance with a distance to the projection reference line, and generate the plurality of pieces of projection data for each projection section of the plurality of projection sections, and
wherein the third generating unit is configured to generate the two-dimensional image for each projection section.

5. The image processing apparatus according to claim 1, wherein the second generating unit is configured to set, when the imaging target object is a breast, a line connecting a papilla and a chest wall in the breast as the projection reference line.

6. The image processing apparatus according to claim 1, wherein the third generating unit is configured to generate, as the two-dimensional image, one of a two-dimensional image obtained by arranging the plurality of pieces of projection data on an arc based on the plurality of projection angles, and a two-dimensional image obtained by arranging the plurality of pieces of projection data in one direction in order of the plurality of projection angles.

7. The image processing apparatus according to claim 1, wherein the second generating unit is configured to perform, as the projection processing, one of processing of calculating a sum of the pixel values, processing of calculating an average of the pixel values, and processing of calculating a maximum value of the pixel values.

8. The image processing apparatus according to claim 1, further comprising an image output unit configured to output the two-dimensional image generated by the third generating unit to a display device.

9. The image processing apparatus according to claim 4, further comprising an image output unit configured to output the two-dimensional image for each projection section, which has been generated by the third generating unit, to a display device in a switching manner for each projection section.

10. The image processing apparatus according to claim 8,
wherein the third generating unit is configured to further generate at least one standard image selected from an axial image, a coronal image, and a sagittal image of the three-dimensional image, and
wherein the image output unit is configured to output, along with the at least one standard image, the two-dimensional image in association with the at least one standard image to the display device.

11. An image processing method comprising:
a first generating step of generating a three-dimensional image with use of an image signal obtained by imaging an imaging target object with a radiation;
a second generating step of setting a projection reference line in the three-dimensional image, and performing projection processing of projecting pixel values of the three-dimensional image to the projection reference line for each projection angle of a plurality of projection angles about the projection reference line, to thereby generate a plurality of pieces of projection data at the plurality of projection angles; and
a third generating step of two-dimensionally arranging the plurality of pieces of projection data based on the plurality of projection angles, to thereby generate a two-dimensional image.

12. A non-transitory computer-readable medium having stored thereon a program for causing, when executed by a computer, the computer to execute the steps of the image processing method of claim 11.
